(19) United States
(12) Patent Application Publication
(10) Pub. No.: US 2018/0360701 A1
(43) Pub. Date: Dec. 20, 2018

(54) STACKED BODY AND SHEET FOR APPLICATION TO SKIN

(71) Applicant: Panasonic Intellectual Property Management Co., Ltd., Osaka (JP)

(72) Inventor: Hirokazu KIMIYA, Kyoto (JP)

(21) Appl. No.: 15/986,951

(22) Filed: May 23, 2018

(30) Foreign Application Priority Data
Jun. 15, 2017 (JP) 2017-117994

(51) Int. Cl. A61K 8/02; A61K 8/65; A61K 8/73; A61K 8/67

(52) U.S. Cl. CPC A61K 8/0212; A61K 8/65; A61K 8/42; A61K 8/67; A61K 8/73

(57) ABSTRACT

Provided is a stacked body including: a fiber substrate layer that contains first fibers; and a fiber assembly that is stacked on the fiber substrate layer, wherein the fiber assembly includes: second fibers that contain a water-soluble first component as a main component and particulates that contain a second component that is capable of forming a hydrogel, and in a case where the second fibers contain the second component, a mass proportion of the second component contained in the particulates is greater than a mass proportion of the second component contained in the second fibers, and an average fiber diameter D1 of the first fibers and an average fiber diameter D2 of the second fibers satisfy the relationship: D1>D2. With the stacked body, embedment of the particulates into the fiber substrate layer is suppressed, and a large number of particulates can be laid in an exposed state.

### TECHNICAL FIELD

The present invention relates to a stacked body that includes a fiber assembly and a fiber substrate, the fiber assembly containing fibers and particulates.

### BACKGROUND ART

Conventionally, various types of sheets for application to the skin that use a hydrogel have been proposed. For example, Patent Literature 1 teaches a sheet in which capsules encapsulating a cosmetic component are carried on a fiber layer. Patent Literature 2 teaches a hydrogel sheet formed by applying a hydrogel that contains a functional component onto a substrate.

### CITATION LIST

### Patent Literatures

[PTL 1] Laid-Open Patent Publication No. 2014-129314

[PTL 2] Laid-Open Patent Publication No. 2010-536810

### SUMMARY OF INVENTION

### Technical Problem

According to Patent Literature 1, capsules are sprayed to nanofibers produced by electrospinning so as to attach the capsules to the outer surface of the nanofibers. In this case, capsules are easily detached from the nanofibers. In addition, Patent Literature 1 also teaches a method in which capsules are mixed with a raw material for nanofibers, and the resulting mixture is electrospun so as to incorporate the capsules in the nanofibers. In this case, the detachment of the capsules is suppressed, but it is difficult to obtain a sufficient effect produced by the use of cosmetic component because the capsules have a small exposed area. Furthermore, the nanofibers are fine and have a small volume, and thus it is not possible to cause the nanofibers to carry or encapsulate a sufficient amount of a functional component. For this reason, it is difficult to obtain a sufficient effect produced by the use of the functional component.

The hydrogel sheet disclosed in Patent Literature 2 is stripped from the substrate before use and transferred onto the skin. Such a sheet is gas impermeable. For this reason, skin respiration may be hindered, or sweat glands may be clogged, while the sheet is in contact with the skin. Accordingly, if the sheet is used for a long period of time, a rash or the like may appear on the skin.

### Solution to Problem

One aspect of the present invention relates to a stacked body including: a fiber substrate layer that contains first fibers; and a fiber assembly that is stacked on the fiber substrate layer, wherein the fiber assembly includes: second fibers that contain a water-soluble first component as a main component and particulates that contain a second component that is capable of forming a hydrogel, and in a case where the second fibers contain the second component, a mass proportion of the second component contained in the particulates is greater than a mass proportion of the second component contained in the second fibers, and an average fiber diameter D1 of the first fibers and an average fiber diameter D2 of the second fibers satisfy the relationship: D1>D2.

Another aspect of the present invention relates to a sheet for application to the skin that includes the above-described stacked body.

### Advantageous Effects of Invention

According to the present invention, embedment of the particulates into the fiber substrate layer is suppressed. Accordingly, a large number of particulates that contain the second component that is capable of forming a hydrogel can be laid in an exposed state in the stacked body.

### BRIEF DESCRIPTION OF DRAWINGS

FIG. 1 is a side view showing an example of a configuration of an electrospinning apparatus that is used to manufacture a stacked body according to one embodiment of the present invention.

FIG. 2 is an electron micrograph of a stacked body obtained in Example 1 (at a magnification of 5,000 times).

FIG. 3 is an infrared absorption spectrum of a fiber assembly obtained in Example 1.

FIG. 4 is an infrared absorption spectrum of particulates obtained in Example 1.

FIG. 5 is an infrared absorption spectrum of enzyme-degraded collagen peptide.

FIG. 6 is an infrared absorption spectrum of sodium hyaluronate.

FIG. 7 is a graph showing a relationship between the mass proportion of sodium hyaluronate and the peak intensity ratio.

FIG. 8A is an electron micrograph of a stacked body obtained in Example 2 (at a magnification of 100 times).

FIG. 8B is an enlarged electron micrograph of FIG. 8A (at a magnification of 500 times)

FIG. 9A is an electron micrograph of a stacked body obtained in Example 3 (at a magnification of 100 times).

FIG. 9B is an enlarged electron micrograph of FIG. 9A (at a magnification of 500 times).

### DESCRIPTION OF EMBODIMENT

### (Stacked Body)

A stacked body according to the present embodiment includes: a fiber substrate layer that contains first fibers; and a fiber assembly that is stacked on the fiber substrate layer. The fiber assembly includes: second fibers that contain a water-soluble first component as a main component, and particulates that contain a second component that is capable of forming a hydrogel.

The average fiber diameter D1 of the first fibers and the average fiber diameter D2 of the second fibers satisfy the relationship: D1>D2. Accordingly, the spacing between second fibers formed in the fiber assembly is likely to be smaller than the spacing between first fibers formed in the fiber substrate layer. For this reason, embedment of the particulates from the fiber assembly into the fiber substrate layer is suppressed. As a result, the area (exposed area) of the particulates that is exposed from the stacked body is increased. Furthermore, a large number of particulates can be carried on the second fibers.

The stacked body described above is useful as a sheet for application to the skin that is used in direct or indirect contact with the skin or by being attached to the skin. This is because the first component and the second component can act on the skin under the presence of water. Such a sheet for application to the skin can be used in the medical field, the aging care field, the child care field, the cosmetic field, and other fields. To be specific, the sheet for application to the skin can be used as a material for medical articles such as adhesive bandages, wound dressings and skin protection sheets, a material for aging care articles such as bedsore prevention sheets, a material for child care articles such as diapers, and a material for cosmetics designed for long-term use.

The sheet for application to the skin that includes the stacked body is used by being brought into contact with the skin such that the fiber assembly opposes the skin. In this case, the first and/or the second component may be, for example, a pharmaceutical component that has a medicinal effect or a cosmetic component that is expected to provide a cosmetic effect, and the first and/or the second component are preferably transdermal. In this case, in a state in which the stacked body is in contact with the skin, the first component (the second fibers) is dissolved due to moisture evaporated from the body or supply of a moisture-containing liquid from the outside, as a result of which the first component and the second component, as well as a third component, which will be described later, can act on the skin.

Hereinafter, the configuration of the stacked body according to the present embodiment will be described specifically by way of an embodiment suitable for use as a sheet for application to the skin. However, the application and configuration of the stacked body is not limited thereto. For example, the first component and the second component may be selected as appropriate according to the application or the like of the stacked body.

### (Fiber Substrate Layer)

The fiber substrate layer functions to support the fiber assembly. The fiber substrate layer contains first fibers, and is constituted by a fiber structure such as, for example, a woven fabric, a knitted fabric, a non-woven fabric, or a felt. Such a fiber substrate layer is highly gas permeable (i.e., has permeability for water vapor, air, oxygen, or the like). For this reason, while the stacked body is in contact with the skin, hindrance to skin respiration and clogging of sweat glands are suppressed. Particularly in the case where the stacked body is used over a long period of time in a state in which it is in contact with the skin, it is important that the fiber substrate layer is gas permeable. Also, because the fiber substrate layer is stretchable, the sheet is suitable for being attached to a movable part of the body.

From the viewpoint of ease of attaining high gas permeability, the fiber substrate layer is preferably a non-woven fabric. There is no particular limitation on the method for producing a non-woven fabric. It is possible to use methods such as a spun bonding method, a dry method (for example, an air laid method), a wet method, a melt blowing method, a needle punching method, an electrospinning method, and the like. The method for producing a non-woven fabric may be selected as appropriate according to the application and the purpose. The fiber substrate layer is preferably manufactured by an electrospinning method, for example, in the case of applications that require a high level of adhesion to the skin, a barrier property against pathogenic substances, harmful substances and irritants, or a waterproof property. This is because, with the electrospinning method, fine fibers can be formed, and the spacing between fibers is likely to be small. Also, in the case of applications where excellent ease of handling and strength are required, or applications where a liquid is supplied from the outside in a state in which the sheet is in contact with the skin, the fiber substrate layer is preferably manufactured by a spun bonding method or the like. This is because with the spun bonding, it is possible to easily form thick fibers.

There is no particular limitation on the average fiber diameter D1 of the first fibers, but the average fiber diameter D1 of the first fibers is preferably 8 μm or less, and more preferably 1 μm or less from the viewpoint of ease of attaining a high level of adhesion to the skin. On the other hand, from the viewpoint of facilitating the support of the fiber assembly, the average fiber diameter D1 is preferably 500 nm or more. As the fiber diameter increases, the spacing between fibers tends to be large. When the average fiber diameter D1 is within the above-described range, the spacing between first fibers is sufficiently large, and thus gas permeability is more likely to be ensured.

As used herein, the term “average fiber diameter D1” refers to an average value of the diameters of the first fibers. Here, the term “diameter of a first fiber” refers to the diameter of a cross section of the first fiber, the cross section being perpendicular to the lengthwise direction of the first fiber. If the cross section does not have a circular shape, the greatest dimension may be taken as the diameter. Alternatively, a width in a direction perpendicular to the lengthwise direction of a first fiber when the fiber substrate layer is viewed from a direction normal to one of the main surfaces of the fiber substrate layer may be taken as the diameter of the first fiber. The average fiber diameter D1 is, for example, an average value of diameters measured at arbitrary locations on arbitrarily selected ten first fibers of the fiber substrate layer. The same applies to the average fiber diameter D2 of the second fibers.

There is no particular limitation on the material of the first fibers as long as it is insoluble in water. It is possible to use, for example, cellulose, rayon, acrylic resin, polypropylene, polyethylene, polyethylene terephthalate, polyamide, polyurethane, cotton, a mixture thereof, or the like. Among these, rayon, cellulose and cotton are preferable because they are flexible and biocompatible, and are also easily available. Also, from the viewpoint of being flexible and ease of control of physical properties such as hydrophilicity, polyurethane is preferable. It is preferable that such preferred materials account for 50 mass % or more of the first fibers.

From the viewpoint of ease of attaining a high level of adhesion to the skin, the mass per unit area of the fiber substrate layer is preferably 200 g/m² or less, and more preferably 80 g/m² or less. On the other hand, from the viewpoint of facilitating the support of the fiber assembly, the mass per unit area of the fiber substrate layer is preferably 2 g/m² or more, and more preferably 10 g/m² or more.

There is no particular limitation on the porosity of the fiber substrate layer, but from the viewpoint of gas permeability, the porosity of the fiber substrate layer is preferably 70 vol % or more, and more preferably 85 vol % or more. On the other hand, from the viewpoint of strength, the porosity of the fiber substrate layer is preferably 95 vol % or less. As used herein, the porosity (vol %) is represented by, for example, (1−mass per unit apparent volume of fiber substrate layer/specific gravity of first fibers)×100.

### (Fiber Assembly)

The fiber assembly includes second fibers that contain a water-soluble first component as a main component, and particulates that contain a second component that is capable of forming a hydrogel. The fiber assembly may be in the form of, for example, a non-woven fabric or cotton. The fiber assembly acts on the skin under the presence of moisture, and it is thereby possible to provide the effects produced by the first component and the second component.

There is no particular limitation on the mass per unit area of the fiber assembly, but considering the effect on the skin, the mass per unit area of the fiber assembly is preferably 10 m g/m² or more, and more preferably 50 m g/m² or more. On the other hand, considering the adhesion to the skin, the mass per unit area of the fiber assembly is preferably 3000 m g/m² or less.

### (Second Fibers)

The second fibers contain a water-soluble first component as a main component (the component that accounts for 50 mass % or more of the second fibers). Accordingly, when the stacked body is brought into contact with the skin under the presence of moisture, the first component (a portion or all of the second fibers) is dissolved due to moisture, and acts on the skin. Furthermore, as a result of dissolution of the first component, the particulates containing the second component can be brought into contact with the skin. The second fibers may contain the second component and/or a third component together with the first component.

From the above viewpoint, it is preferable that the second fibers contain water together with the first component. This is because when the stacked body is brought into contact with the skin, the stacked body can be easily caused to adhere to the skin without supply of moisture. Also, as a result of the second fibers containing water, dryness of the skin is suppressed while the stacked body is in contact with the skin.

However, after the stacked body has been brought into contact with the skin, moisture may be supplied as appropriate by using a spray so as to promote the dissolution of the first component. Alternatively, a liquid containing a component (the first component, the second component, a third component, which will be described later, and the like) that can exhibit a desired action on the skin may be further supplied to the stacked body. By selecting a fiber material suitable for the supplied liquid, liquid permeability can be imparted to the fiber substrate layer in addition to the gas permeability. Also, by supplying moisture at the time when the stacked body is stripped, the stacked body is easily stripped from the skin.

The spacing between second fibers formed in the fiber assembly is likely to be smaller than the spacing between first fibers formed in the fiber substrate layer. When the stacked body is viewed from the fiber assembly side, the second fibers are laid such that the second fibers extend across the spacing between first fibers formed in the fiber substrate layer. With this configuration, embedment of the particulates from the fiber assembly into the fiber substrate layer is suppressed. Accordingly, when an arbitrary region R (for example, a region that includes ten or more particulates) in the stacked body is viewed from the fiber assembly side, the number of particulates that overlap first fibers and are present in front of the first fibers is greater than the number of particulates that overlap first fibers and are present behind the first fibers. Accordingly, the particulates can effectively act on the skin.

Also, because the average fiber diameter D2 is small, the exposed area of the particulates is increased, and the adhesion of the stacked body to the skin is increased. In particular, the average fiber diameter D1 and the average fiber diameter D2 preferably satisfy the relationship: D1>D2×5, and more preferably satisfy the relationship: D1>D2×20.

The average fiber diameter D2 of the second fibers is preferably 500 nm or less, more preferably 200 nm or less, and even more preferably 100 nm or less. This allows the second fibers to be easily dissolved, and the adhesion to the skin is increased. On the other hand, in order to cause the first component to act on the skin in an amount that is sufficient to exhibit its effect, the average fiber diameter D2 is preferably 20 nm or more, and more preferably 50 nm or more.

The spacing between fibers can be reduced as the fiber diameter is reduced. For this reason, when the average fiber diameter D2 is within the above-described range, the spacing between second fibers is sufficiently small, and embedment of the particulates into the fiber substrate layer is more likely to be suppressed. Also, a good texture can be obtained. In addition, a capillary phenomenon occurs between second fibers, and thus the fiber assembly can easily absorb moisture. Accordingly, the first component is more easily dissolved.

It is preferable that a plurality of second fibers are bonded to the surface of particulates so as to support the particulates. In this case, embedment of the particulates into the fiber substrate layer is more likely to be suppressed, and detachment of the particulates from the fiber assembly is suppressed. Also, as a result of particulates being supported by a plurality of second fibers, the particulates are easily laid on the vicinity of the surface of the fiber assembly that is opposed to the skin, rather than the vicinity between the interface between the fiber substrate layer and the fiber assembly. In this case, when the region R in the stacked body is viewed from the fiber assembly side, for example, the number of second fibers that overlap particulates and are present in front of the particulates is smaller than the number of second fibers that overlap particulates and are present behind the particulates. Accordingly, the particulates can more easily act on the skin. Note that the second fibers and the particulates are in point contact or line contact, and thus the particulates have a large exposed area.

Here, spindle-shaped bulges (hereinafter, referred to as “beads”) may be formed in the second fibers. Unlike the particulates, the beads are formed mainly by the first component that was not sufficiently drawn and thus was not formed into the second fibers during the process of electrospinning. With the beads, the adhesion of the stacked body to the skin is improved. Furthermore, with the beads, the duration required for dissolution of the first component increases. Accordingly, it is possible to control the duration of the action of each component contained in the stacked body. In the case where the second fibers contain beads as described above, the diameter may be measured by avoiding bead portions, and then the average fiber diameter D2 may be calculated. The beads may contain, together with the first component, the second component and/or a third component.

There is no particular limitation on the size of the beads. In the case where each component contained in the fiber assembly is caused to act over a long period of time, it is preferable that a plurality of beads are provided on a single fiber. With this configuration, the duration required for dissolution of the fibers can be further increased.

The average diameter D4 of the beads is an average value of the greatest diameters of a plurality of (for example, ten) beads. The greatest diameter of a bead refers to the greatest dimension of the bead at which the outline of the bead is clearly visible when the fiber assembly is viewed from one direction. The greatest diameter of a bead can be determined, for example, in the manner described below. On a SEM micrograph of the fiber assembly, the fiber diameter of a single second fiber is measured while moving toward a bead on the single second fiber, and a spot where the fiber diameter first reaches two times or more the average fiber diameter D2 is defined as end portion T1 that is one end portion of the bead. Then, the fiber diameter of the same second fiber is measured from the opposite side of the same bead while moving toward the bead on the second fiber, and a spot where the fiber diameter first reaches two times or more the average fiber diameter D2 is defined as end portion T2 that is the other end portion of the bead. A straight line that connects the end portion T1 and the end portion T2 is drawn, and the greatest length of the bead in a direction perpendicular to the straight line is referred to as the greatest diameter of the bead.

The first component is a component that is water soluble and does not form a hydrogel. The first component may be, for example, any of collagens. Examples of the collagens include collagen, collagen peptide, gelatin, and the like. From the viewpoint of providing a water solubility and ease of forming second fibers, the first component preferably has a weight average molecular weight of 500 to 80,000, and more preferably 1,000 to 40,000.

### (Particulates)

The particulates contain a second component that is capable of forming a hydrogel. Accordingly, when the stacked body is brought into contact with the skin under the presence of moisture, the particulates containing the hydrogel adhere to the skin, and thus can act directly on the skin. From this viewpoint, it is preferable that the particulates contain water together with the second component. This is because when the stacked body is brought into contact with the skin, the particulates can adhere to the skin without supply of moisture. Furthermore, because the particulates have water retention properties, dryness of the skin can be suppressed over a long period of time. Also, as described above, moisture may be supplied after the stacked body has been brought into contact with the skin. In this case, the particulates can incorporate and retain the supplied moisture.

It is preferable that particulates are included in the fiber assembly in a supported state by a plurality of second fibers. The particulates contain a second component that is capable of forming a hydrogel. In the case where the second fibers contain the second component, the mass proportion R_{2P} of the second component contained in the particulates is greater than the mass proportion R_{2F} of the second component contained in the second fibers. The mass proportion R_{2P}-to-mass proportion R_{2F} ratio (R_{2P}/R_{2F}) is, for example, 2 to 20. The mass proportion R_{2P} is, for example, 20 to 80 mass %. Also, from the viewpoint of allowing the second component to easily exhibit its effect, it is preferable that the second component is a main component of the particulates that accounts for 50 mass % or more of the particulates excluding moisture. The particulates may contain the first component and/or a third component.

The average particle size D3 of the particulates is preferably 0.2 to 20 μm, and more preferably 0.5 to 10 μm. By doing so, embedment of the particulates into the fiber substrate layer is unlikely to occur, and the amount of the second component contained in the fiber assembly can be increased. Furthermore, the particulates can more easily come into contact with the skin.

The average particle size D3 of the particulates is an average value of the greatest diameters of a plurality of (for example, ten) particulates. The greatest diameter of a particulate refers to the greatest dimension of the particulate at which the outline of the particulate is clearly visible when the fiber assembly is viewed from one direction.

The particulates are required to be incorporated as much as possible in the fiber assembly from the viewpoint of allowing the particulates to easily act on the skin. For example, the mass proportion of the particulates to the fiber assembly is preferably 5 to 40 mass %, and more preferably 10 to 25 mass %. Because the particulates are not easily embedded into the fiber substrate layer, the particulates can be incorporated in the fiber assembly in a proportion described above.

It is preferable that the average fiber diameter D2 of the second fibers and the average particle size D3 of the particulates satisfy the relationship: D2<D3. In this case, the spacing between second fibers is likely to be smaller than a particulate, and thus the particulate can be easily supported by a plurality of second fibers. Accordingly, embedment of the particulates into the fiber substrate layer is more likely to be suppressed. Furthermore, in this case, because the exposed area of the particulates is increased, the particulates can more easily come into contact with the skin. In particular, the average fiber diameter D2 of the second fibers and the average particle size D3 of the particulates preferably satisfy the relationship: D2<D3× 1/20, and more preferably satisfy the relationship: D2<D3× 1/50.

The average fiber diameter D1 of the first fibers and the average particle size D3 of the particulates may satisfy the relationship: D1<D3. In this case, the spacing between first fibers is likely to be smaller than a particulate, and thus even if the particulates are embedded into the fiber substrate layer, detachment of the particulates from the fiber substrate layer to the outside is more likely to be suppressed. In particular, the average fiber diameter D1 of the first fibers and the average particle size D3 of the particulates preferably satisfy the relationship: D1<D3×½, and more preferably satisfy the relationship: D1<D3×⅕.

It is preferable that the surface of a particulate has irregularities formed thereon. This is to increase the specific surface area of the particulate. With this configuration, the contact area of the particulate with moisture increases, and thus the particulate can easily exhibit its water retention performance. There is no particular limitation on the shape and height of the irregularities.

The second component may be, for example, at least one selected from the group consisting of a hyaluronic acid salt, a hyaluronic acid derivative, a water-soluble alginic acid salt, and an alginic acid derivative. Examples of cations that form salts include sodium ions, potassium ions, magnesium ions, ammonium ions, calcium ions, and the like. Note, however, that alginic acid salts formed by divalent cations (for example, calcium ions) other than magnesium ions are not included in the second component because they are not water soluble. Examples of derivatives include esters, acetylated products, and the like. The second component may be used singly or in a combination of two or more.

Among these, from the viewpoint of providing a moisturizing effect, the second component is preferably a hyaluronic acid salt. As a result of a hyaluronic acid salt being contained as the second component, it is possible to impart a moisturizing effect to the fiber assembly. Also, from the viewpoint of providing a hemostasis effect, the second component is preferably a salt formed by calcium ions. As a result of a calcium salt being contained as the second component, it is possible to impart a hemostasis effect to the fiber assembly.

The fibers and/or the particulates may contain a functional component (third component) other than the first component (for example, any of collagens) and the second component (for example, at least one selected from the group consisting of a hyaluronic acid salt, a hyaluronic acid derivative, a water-soluble alginic acid salt, and an alginic acid derivative).

The third component may be water soluble, less water soluble, or water dispersible. Also, the third component may be a substance that is capable of forming a hydrogel, or a substance that does not form a hydrogel. The third component may be, for example, a pharmaceutical component that has a medicinal effect, a cosmetic component that is expected to provide a cosmetic effect, or an adjusting component that adjusts the properties of a raw material liquid, which will be described later. Examples of the pharmaceutical component include a hemostatic agent, an antiphlogistic agent, an autoinducer inhibitor, a transdermal pharmaceutical product, and the like. Examples of the cosmetic component include a vitamin C derivative, lactic acid, malic acid, a malic acid salt or derivative, tartaric acid, a tartaric acid salt or derivative, citric acid, a citric acid salt or derivative, sericin, a perfume, and the like. Examples of the adjusting component include a thickener, an antiseptic agent, a pH adjusting agent, an electroconductivity adjusting agent, and the like. The third component may be used singly or in a combination of two or more.

The third component may be incorporated more in the fibers, or may be incorporated more in the particulates depending on the level of compatibility with the first component and the second component, the solubility in water, or the like. For example, a third component that is highly compatible with the first component is likely to be incorporated in the fibers. It is preferable that a pharmaceutical component and/or a cosmetic component are/is incorporated in the fibers as the third component because the component(s) can act on the skin in a short time by dissolution of the first component. The dissolution of the first component is controlled by adjusting, for example, the supply of moisture or the humidity of the surroundings.

On the other hand, a third component that is highly compatible with the second component is likely to be incorporated in the particulates. It is preferable that a pharmaceutical component and/or a cosmetic component are/is incorporated in the particulates as the third component because the component(s) can act on the skin over a long period of time.

It is preferable that the fibers and/or the particulates contain a pH adjusting agent as the third component. Accordingly, the pH level of the fibers and/or the particulates is controlled. Here, the performance of the function of the pharmaceutical component and the cosmetic component may be dependent on the pH level. For this reason, in the case where the fibers and/or the particulates contain at least one of a pharmaceutical component and a cosmetic component as the third component, by further adding a pH adjusting agent as the third component, the effect of the pharmaceutical component and/or the cosmetic component is more easily exerted.

Examples of the pH adjusting agent include: acids such as citric acid, acetic acid, phosphoric acid, sulfuric acid, gluconic acid, and succinic acid; carbonates such as potassium carbonate and sodium hydrogencarbonate; sodium hydroxide; potassium hydroxide; and the like. It is preferable that the components listed above are used in the form of a buffer solution that contains a salt thereof (for example, a phosphoric acid buffer solution, a citric acid buffer solution, an acetic acid buffer solution, or the like) so as to stabilize the pH level.

The stacked body may include a third layer in addition to the fiber substrate layer and the fiber assembly.

For example, in the case where the average fiber diameter D1 of the first fibers contained in the fiber substrate layer is too large, a third layer (intermediate layer) may be provided between the fiber assembly and the fiber substrate layer. The third layer may be composed of fibers that have an average fiber diameter smaller than the average fiber diameter D1 and may be greater than the average fiber diameter D2. There is no particular limitation on the material of the intermediate layer, and the material of the intermediate layer may be the same as that of the fiber substrate layer.

Also, a decorative third layer (cover layer) may be provided on the outermost surface of the fiber substrate layer. There is no particular limitation on the material and form of the cover layer as long as it does not hinder the gas permeability of the fiber substrate layer. It is possible to use, for example, a moisture-permeable waterproof nonwoven fabric, film or the like. Alternatively, in order to improve ease of handling, and suppress detachment of the particulates or damage to the fiber assembly before use, a third layer (protection layer) may be provided on the outermost surface of the fiber assembly. As the protection layer, it is possible to use a film or the like that has releasability.

### (Method for Manufacturing Stacked Body)

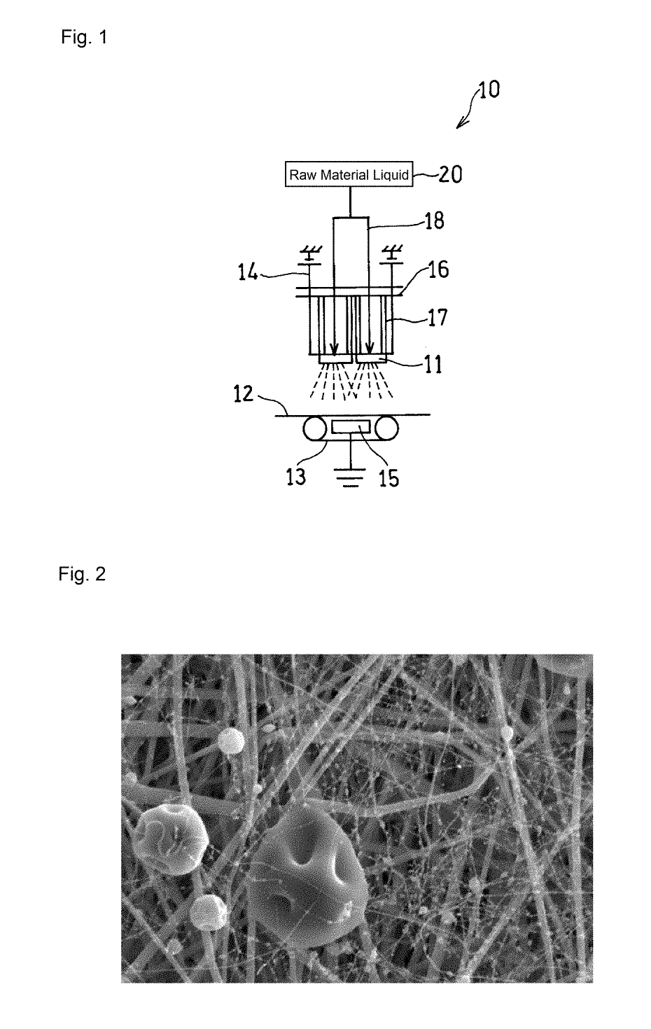

Hereinafter, a method for manufacturing a stacked body will be described specifically with reference to the drawings, but the method for manufacturing a stacked body is not limited thereto. FIG. 1 is a side view showing an example of a configuration of an electrospinning apparatus that is used to manufacture a fiber assembly. A similarly configured apparatus can be used to manufacture a fiber substrate layer by an electrospinning method.

The stacked body is manufactured by a method that includes: for example, a preparation step of preparing a raw material liquid that contains a water-soluble first component, a second component that is capable of forming a hydrogel, and water; and an electrospinning step of producing second fibers containing the first component as a main component and particulates containing the second component from the raw material liquid by an electrospinning method so as to deposit the second fibers and the particulates on a fiber substrate layer. According to this method, it is possible to obtain a fiber assembly in which at least a portion of the particulates are supported by a plurality of second fibers.

In an electrospinning method, a target is prepared and grounded or negatively (or positively) charged, and a raw material liquid (normally, a solution in which a raw material for fibers are dissolved) to which a positive (or negative) potential has been applied is discharged toward the target through a nozzle. The solvent contained in the raw material liquid is volatilized before it reaches the target, and an assembly of fibers produced by an electrostatic drawing phenomenon is deposited on the target.

Here, the raw material liquid contains a water-soluble first component, a second component that forms a hydrogel, and water. When the raw material liquid is electrostatically drawn, at least a portion of water contained in the raw material liquid is removed (evaporated). As a result, the first component forms second fibers. The viscosity of the raw material liquid may be increased due to the inclusion of the second component. For this reason, the second fibers are easily formed. On the other hand, the concentration of the second component is increased, and the second component forms a gel structure and turns into particulates as a result of being released from the discharge pressure. During this process, a portion of the first component may be introduced into the gel structure. However, in the case where the second fibers contain the second component, the mass proportion R_{2P} of the second component contained in the particulates is greater than the mass proportion R_{2F} of the second component contained in the fibers.

As described above, because particulates containing the second component and second fibers containing the first component are produced within the same space, the particulates and the second fibers come into contact with and bond to each other. After that, the second fibers that are bonded to the particulates are deposited on the target (for example, the fiber substrate layer), and a fiber assembly is formed. The difference in spinnability between the first component and the second component is considered to result from the differences in the molecular weight, the solubility in solvents, the surface tension, the intermolecular interaction, and the like.

### (Preparation Step)

First, a raw material liquid 20 that contains a first component, a second component and water is prepared. In the raw material liquid 20, the first component is dissolved, and the second component is dissolved or dispersed.

The concentration of the first component in the raw material liquid 20 is not particularly limited and may be set as appropriate by taking into consideration the viscosity of the raw material liquid 20, or the like. In terms of the formability of second fibers, the concentration of the first component in the raw material liquid 20 is preferably 5 to 40 mass %, and more preferably 10 to 25 mass %. The concentration of the second component in the raw material liquid 20 is not particularly limited and may be set as appropriate as long as the concentration is within a range that does not cause the raw material liquid 20 to be gelled. In particular, from the viewpoint of ease of forming particulates, the concentration of the second component in the raw material liquid 20 is preferably 0.01 to 5 mass %, and more preferably 0.1 to 2 mass %.

The viscosity of the raw material liquid 20 may be set as appropriate so as to be suitable for the electrospinning method. In particular, the raw material liquid 20 preferably has a viscosity of 500 to 30,000 m Pa·s, and more preferably 1,000 to 15,000 m Pa·s. The viscosity is measured under conditions at 25° C. with the use of a rotational viscometer at a shear rate of 1 s⁻¹. When the raw material liquid 20 has a viscosity within the above-described range, stable electrospinning is possible, and the particulates are uniformly laid on the target with ease. The second component has the effect of increasing the viscosity of the raw material liquid 20, and thus the viscosity of the raw material liquid 20 can be controlled by blending the second component. However, the raw material liquid 20 may contain other components for adjusting the viscosity.

The raw material liquid 20 may contain a solvent (hereinafter, referred to as “second solvent”) other than water. There is no particular limitation on the second solvent as long as it is compatible with water. The second solvent may be selected as appropriate according to the types of the first component and the second component, the manufacturing conditions, and the like. In particular, from the viewpoint of excellent compatibility with water and excellent volatility, the second solvent is preferably any of alcohols including methanol, ethanol, 1-propanol, 2-propanol, isobutyl alcohol, and hexafluoro isopropanol. These may be used singly or in a combination of two or more. However, from the viewpoint of ensuring solubility of the first component, the proportion of the second solvent in the total amount of the solvents is preferably less than 50 mass %, and more preferably less than 20 mass %.

Also, in the case where the first component is collagen peptide, and the second component is at least one of a hyaluronic acid salt and a water-soluble alginic acid salt, the raw material liquid 20 preferably contains a third component other than the first component and the second component. In this case, the third component is contained in the fibers and/or the particulates. According to the present embodiment, with a very simple operation of blending a functional component in the raw material liquid 20, it is possible to cause the fiber assembly to retain various types of functional components in a less detachable manner.

In the case where the raw material liquid 20 contains a pH adjusting agent as the third component, the viscosity of the raw material liquid 20 can be easily adjusted to a viscosity level suitable for spinning. This is because the solubility of collagen peptide, a hyaluronic acid salt and a water-soluble alginic acid salt in water is dependent on the pH level.

There is no particular limitation on the concentration of the third component in the raw material liquid 20 as long as the concentration is within a range that does not hinder the formation of fibers and particulates. The concentration of the third component may be set as appropriate by taking into consideration the function of the third component. The concentration of the third component is preferably, for example, 0.01 to 5 mass %, and more preferably 0.1 to 2 mass %.

### (Electrospinning Step)

An electrospinning apparatus 10 used in electrospinning includes, for example, discharging units 11 for discharging a raw material liquid 20, a charging means that positively charges the discharged raw material liquid 20, and a conveyor belt 13 that supports a target 12. The conveyor belt 13 functions, together with the target 12, as a collector unit that collects a fiber assembly.

Here, a fiber substrate layer is used as the target 12. This makes it possible to manufacture a stacked body in a single step. However, the target 12 may be anything other than the fiber substrate layer. In this case, a fiber assembly deposited on the target 12 may be removed from the target 12, and then stacked on a fiber substrate layer.

Each discharging unit 11 is made of a conductor, has an elongated shape, and is internally provided with a hollow portion. The hollow portion serves as a housing portion that houses the raw material liquid 20. A plurality of discharge outlets (not shown) for discharging the raw material liquid 20 are provided in a plurality of locations on the side of the discharging unit 11 that opposes the target 12. The distance between the discharge outlets of the discharging unit 11 and the target 12 may be, for example, 100 to 600 mm although it depends on the scale of the electrospinning apparatus 10 as well as the desired fiber diameter and particle size.

The raw material liquid 20 is supplied to the hollow portions of the discharging units 11 through pipes 18 by the pressure of a pump (not shown) that is in communication with the hollow portions of the discharging units 11, and discharged toward the target 12 through the discharge outlets. The discharged raw material liquid 20 in a charged state causes an electrostatic explosion while moving through a space (production space) between the discharging units 11 and the target 12 so as to produce second fibers that contain the first component and particulates that contain the second component. The produced second fibers and the particulates supported by the second fibers are deposited on the target 12, thereby forming a fiber assembly. The amount of deposited fiber assembly, the average fiber diameter D2 of the second fibers and the average particle size D3 of the particulates are controlled by adjusting the pressure at which the raw material liquid 20 is discharged, the applied voltage, the composition of the raw material liquid 20, the concentration of the raw materials of the raw material liquid 20, and the environment (environmental composition, temperature, humidity, pressure and the like) of the production space.

The charging means for charging the discharging units 11 and the target 12 are constituted by a voltage application apparatus 14 for applying voltage to the discharging units 11 and a counter electrode 15 that is provided in parallel to the conveyor belt 13. The counter electrode 15 is earthed (grounded). Accordingly, a potential difference that corresponds to the voltage applied by the voltage application apparatus 14 can be generated between the discharging units 11 and the counter electrode 15 (the target 12). There is no particular limitation on the configuration of the charging means. For example, the target 12 may be negatively charged. Also, instead of providing the counter electrode 15, the conveyor belt 13 may be made of a conductor.

Above the discharging units 11, a first supporting unit 16 parallel to the target 12 is installed. The discharging units 11 are supported by, for example, a second supporting unit 17 extending downward from the first supporting unit 16 such that the longitudinal direction of the discharging units 11 is parallel to the main surface of the target 12. The first supporting unit 16 may be movable such that it can pivotally move the discharging unit 11.

The electrospinning apparatus 10 is not limited to the configuration described above. For example, each discharging unit 11 may have a cross section whose shape gradually tapers from its upper end toward its lower end (V-shaped nozzle), the cross section being perpendicular to the longitudinal direction of the discharging unit 11. Also, the discharging unit 11 may include one or more needle shaped nozzles.

After the electrospinning step, the adjustment of water content or the removal of the solvents contained in the fibers and/or the particulates may be performed by air drying, decompression, or heating under conditions that do not cause damage to the fiber assembly. The water content may affect, in addition to the softness and texture of the fiber assembly, the action of each component, the storage properties of the fiber assembly, and the like.

### EXAMPLES

Hereinafter, the present invention will be described in further detail by way of examples. However, it is to be noted that the present invention is not limited to the examples given below.

### Example 1

### (1) Preparation of Raw Material Liquid

A raw material liquid (with a viscosity at a shear rate of 1 s⁻¹ of 10.5 Pa·s) was obtained by mixing and dissolving, in ultrapure water, sodium hyaluronate (Na hyaluronate) and collagen peptide (enzyme-degraded collagen peptide with an average molecular weight of 2,000) so as to achieve a Na hyaluronate concentration of 1.5 mass % and a collagen peptide concentration of 10 mass %.

### (2) Formation of Stacked Body

A stacked body was obtained by electrospinning the obtained raw material liquid onto a non-woven fabric with an applied voltage of 45 kV. A fiber assembly formed on the non-woven fabric contained second fibers having an average fiber diameter D2 of 60 nm and particulates having an average particle size D3 of about 2.5 μm. The non-woven fabric had a thickness of 60 μm and a mass per unit area of 9.4 g/m², and the first fibers had an average fiber diameter D1 of 550 nm and contained 50 mass % of polyurethane and 50 mass % of polyether sulphone.

A scanning electron microscope (SEM) micrograph of the obtained stacked body is shown in FIG. 2. FIG. 2 is a micrograph captured from one of the main surfaces of the stacked body at a magnification of 5,000 times. As can be seen from FIG. 2, the second fibers are provided so as to extend across the spacing between first fibers formed in the fiber substrate layer. With this configuration, embedment of the particulates into the fiber substrate layer is suppressed. Furthermore, at least a portion of the particulates are supported by a plurality of second fibers, and irregularities are formed on the surface of the particulates.

### (3) Determination of Mass Proportions of Components in Fiber Assembly

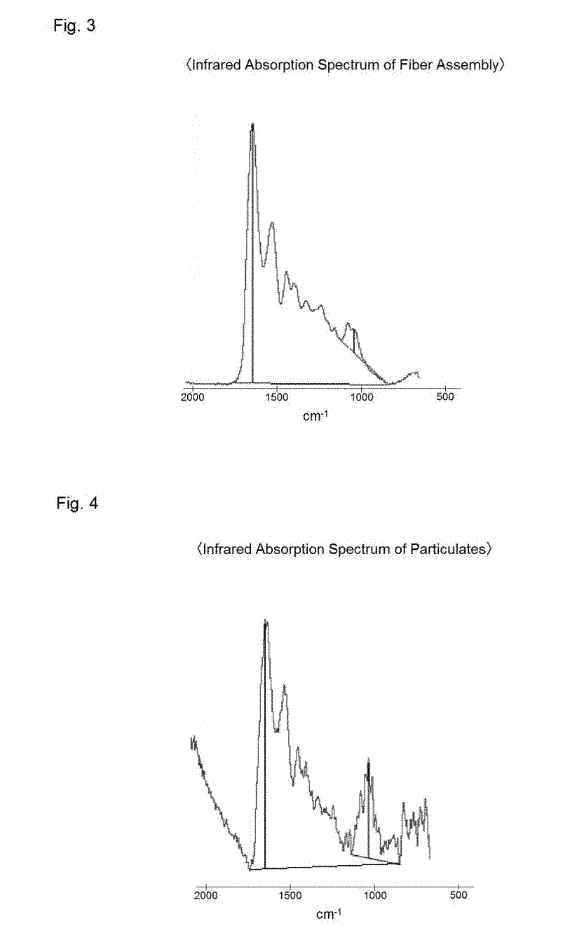

A fiber assembly was produced separately under the same spinning conditions as described in (2) above so as to determine the mass proportions of components contained in the fiber assembly. An infrared absorption spectrum of the entire fiber assembly obtained was acquired by a KBr method using a microscopic infrared absorption measurement apparatus (Nicolet 6700 available from ThermoFisher Scientific, Inc.) (FIG. 3). Also, three particulates having a particle size of 2 μm were taken out from the fiber assembly with the use of a manipulator (AXIS-PRO available from Micro Support, Co., Ltd.), and were placed on a KBr plate. After that, an infrared absorption spectrum was acquired in the same manner (FIG. 4).

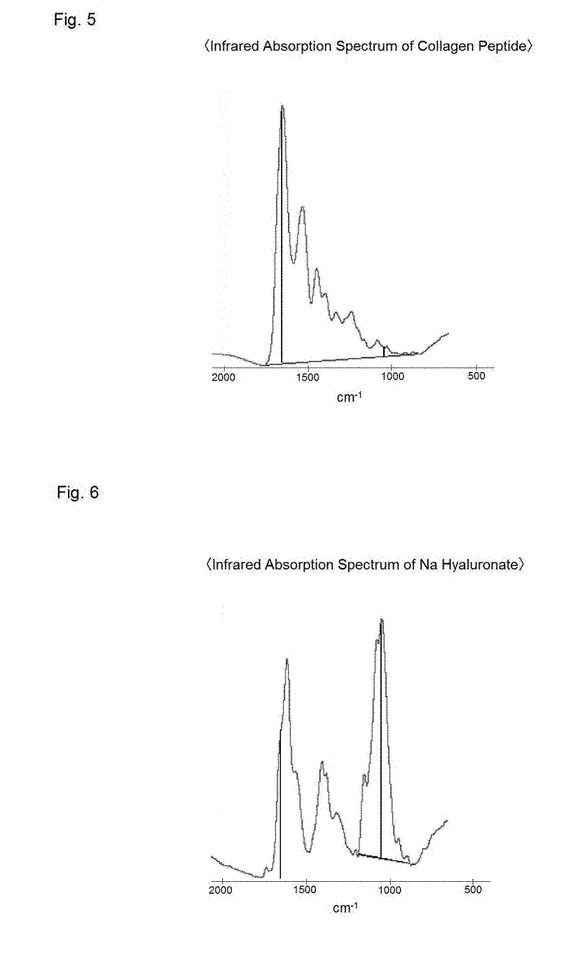

Meanwhile, the above-described collagen peptide and Na hyaluronate powders were placed on KBr plates, respectively, and infrared absorption spectrums that serve as criteria (reference spectrums) were acquired in the same manner as described above. The infrared absorption spectrum of collagen peptide is shown in FIG. 5, and the infrared absorption spectrum of Na hyaluronate is shown in FIG. 6. Collagen peptide had a characteristic absorption peak at 1650 cm⁻¹, and Na hyaluronate had a characteristic absorption peak at 1050 cm⁻¹.

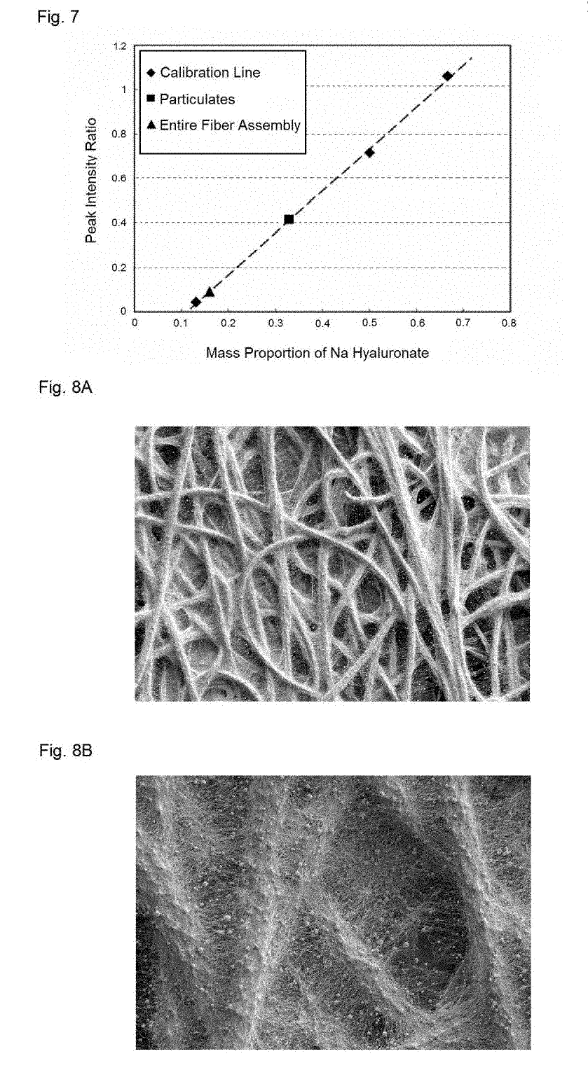

Furthermore, aqueous solutions were prepared by mixing collagen peptide and Na hyaluronate with water so as to achieve a mass ratio of collagen peptide to Na hyaluronate of 67:33, 50:50 and 13:87. Each aqueous solution was applied onto an aluminum foil and dried so as to form a thin film. Then, an infrared absorption spectrum was acquired by using a reflection method under the same conditions as described above. From the infrared absorption spectrum thus acquired and the reference spectrums, a peak intensity ratio was calculated at each of the above-described absorption peaks, and the obtained peak intensity ratios and the mass proportion of Na hyaluronate in the thin film were plotted on a graph. Then, as shown in FIG. 7, a calibration line was drawn based on the plotted points, and the relationship between the peak intensity ratio and the mass proportion of Na hyaluronate was estimated.

The peak intensity ratio calculated from the infrared absorption spectrum of the particulates and the reference spectrums was plotted on the above graph, and the mass proportion R_{2P} of Na hyaluronate contained in the particulates was determined and found to be about 33 mass %. A mass proportion R₂ of Na hyaluronate contained in the entire fiber assembly was determined in the same manner and found to be about 15 mass %. From the calculation results and the mass proportion of the particulates to the fiber assembly, it can be seen that the mass proportion R_{2P} of Na hyaluronate (the second component) contained in the particulates is greater than the mass proportion R_{2F} of Na hyaluronate contained in the fibers. However, it is considered that the actual proportion of the second component in the particulates is greater than the above value calculated from the infrared absorption spectrums because fibers adhering to the particulates or fibers that are present around the particulates are also taken out when the particulates are taken out.

Also, from the above conclusion that the mass proportion R_{2P} of Na hyaluronate contained in the particulates is greater than the mass proportion R₂ of Na hyaluronate contained in the entire fiber assembly, it can be said that the mass proportion R_{2F} of Na hyaluronate contained in the fibers is smaller than the mass proportion R₂ of Na hyaluronate contained in the entire fiber assembly. That is, the mass proportion R_{2F} of Na hyaluronate contained in the fibers is less than 15 mass % (<mass proportion R₂), and the remainder (85 mass % or more) of the fibers is collagen peptide. That is, the main component (the component that accounts for 50 mass % or more of the fibers) of the fibers is collagen peptide.

### Example 2

A stacked body was obtained in the same manner as in Example 1, except that a medical surgical tape (Micropore available from 3M Corporation, a rayon non-woven fabric with an average fiber diameter D1 of 20 μm) was used as a fiber substrate layer. A fiber assembly formed on the medical surgical tape contained second fibers having an average fiber diameter D2 of 60 nm and particulates having an average particle size D3 of about 1 m.

SEM micrographs of the obtained stacked body are shown in FIGS. 8A and 8B. FIG. 8A is a micrograph captured from one of the main surfaces of the stacked body at a magnification of 100 times, and FIG. 8B is an enlarged micrograph obtained by capturing the same portion as that of FIG. 8A at an increased magnification of 500 times. As can be seen from FIGS. 8A and 8B, a large number of particulates are laid in an exposed state on a fiber substrate layer that has a large fiber spacing. That is, it can be seen that second fibers are provided so as to extend across interstices between first fibers that form the fiber substrate layer. The second fibers are finer than the first fibers, and particulates are provided so as to be supported by the second fibers. Embedment of the particulates into the fiber substrate layer is thereby suppressed.

### Example 3

A raw material liquid (with a viscosity at a shear rate of 1 s⁻¹ of 13.1 Pa·s) was prepared in the same manner as in Example 1, except that Na hyaluronate and collagen peptide were mixed and dissolved in a phosphoric acid buffer solution (with a pH of 7.4 and a concentration of 10 mM) so as to achieve a Na hyaluronate concentration of 1.5 mass % and a collagen peptide concentration of 10 mass %, and a stacked body was obtained by forming a fiber assembly on a medical surgical tape that is the same as that used in Example 2. The fiber assembly formed on the medical surgical tape included second fibers having an average fiber diameter D2 of 60 nm and particulates having an average particle size D3 of about 1 m. The phosphoric acid buffer solution was prepared by dissolving a predetermined amount of pH adjusting agents (sodium dihydrogen phosphate dihydrate and disodium hydrogen phosphate) in ultrapure water.

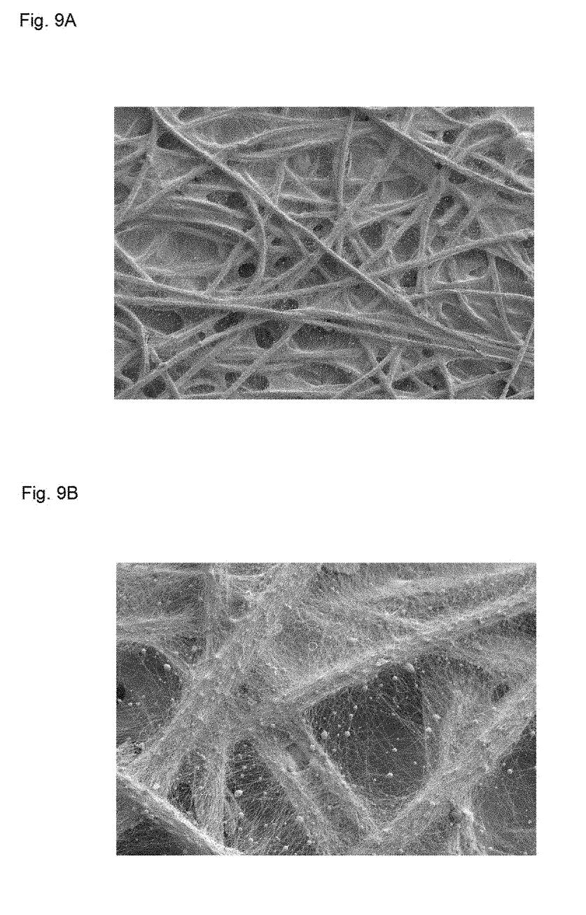

SEM micrographs of the obtained stacked body are shown in FIGS. 9A and 9B. FIG. 9A is a micrograph captured from one of the main surfaces of the stacked body at a magnification of 100 times, and FIG. 9B is an enlarged micrograph obtained by capturing the same portion as that of FIG. 9A at an increased magnification of 500 times. As can be seen from FIGS. 9A and 9B, a large number of particulates are laid in an exposed state on a fiber substrate layer that has a large fiber spacing. That is, it can be seen that second fibers are provided so as to extend across interstices between first fibers that form the fiber substrate layer. The second fibers are finer than the first fibers, and particulates are provided so as to be supported by the second fibers. Embedment of the particulates into the fiber substrate layer is thereby suppressed.

### INDUSTRIAL APPLICABILITY

With the stacked body according to the present invention, embedment of the particulates into the fiber substrate layer is suppressed, and a large number of particulates can be provided in an exposed state in the stacked body. Accordingly, the stacked body according to the present invention can be used in various types of applications such as a sheet for application to the skin.

### DESCRIPTION OF REFERENCE SIGNS

10: Electrospinning Apparatus
11: Discharging Unit
12: Target
13: Conveyor Belt
14: Voltage Application Apparatus
15: Counter Electrode
16: First Supporting Unit
17: Second Supporting Unit
18: Pipe
20: Raw Material Liquid

1. A stacked body comprising:
a fiber substrate layer that contains first fibers; and
a fiber assembly that is stacked on the fiber substrate layer,
wherein the fiber assembly includes: second fibers that contain a water-soluble first component as a main component and particulates that contain a second component that is capable of forming a hydrogel, and
in a case where the second fibers contain the second component, a mass proportion of the second component contained in the particulates is greater than a mass proportion of the second component contained in the second fibers, and
an average fiber diameter D1 of the first fibers and an average fiber diameter D2 of the second fibers satisfy the relationship: D1>D2.

2. The stacked body in accordance with claim 1,
wherein the average fiber diameter D2 of the second fibers and an average particle size D3 of the particulates satisfy the relationship: D2<D3.

3. The stacked body in accordance with claim 2,
wherein the average fiber diameter D1 of the first fibers and the average particle size D3 of the particulates satisfy the relationship: D1<D3.

4. The stacked body in accordance with claim 1,
wherein at least a portion of the particulates are supported by the second fibers.

5. The stacked body in accordance with claim 1,
wherein the average fiber diameter D2 of the second fibers is 500 nm or less.

6. The stacked body in accordance with claim 1,
wherein the average particle size D3 of the particulates is 0.2 m or more.

7. A sheet for application to the skin comprising the stacked body in accordance with claim 1.

8. The sheet for application to the skin in accordance with claim 7,
wherein the first component is any of collagens.

9. The sheet for application to the skin in accordance with claim 7,
wherein the second component is at least one selected from the group consisting of a hyaluronic acid salt, a hyaluronic acid derivative, a water-soluble alginic acid salt, and an alginic acid derivative.

10. The sheet for application to the skin in accordance with claim 7,
wherein the first component is any of collagens,
the second component is at least one selected from the group consisting of a hyaluronic acid salt, a hyaluronic acid derivative, a water-soluble alginic acid salt, and an alginic acid derivative, and
at least either of the second fibers and the particulates further contain a third component other than the first component and the second component.
